(19) United States
(12) Patent Application Publication
(10) Pub. No.: US 2023/0074628 A1
(43) Pub. Date: Mar. 9, 2023

(54) SYSTEM AND METHOD FOR AUTOMATING BEDSIDE INFECTION AUDITS USING MACHINE LEARNING

(71) Applicant: Karan Syal, Newark, CA (US)

(72) Inventors: Karan Syal, Newark, CA (US); Vindhya Koppaka, San Jose, CA (US)

(73) Assignee: cAST Technologies Inc., Milpitas, CA (US)

(21) Appl. No.: 17/940,793

(22) Filed: Sep. 8, 2022

Related U.S. Application Data
(60) Provisional application No. 63/242,063, filed on Sep. 9, 2021.

(51) Int. Cl. A61B 5/00; G06N 20/00; G16H 10/40; G16H 10/60

(52) U.S. Cl. CPC A61B 5/445; G06N 20/00; A61B 5/002; G16H 10/40; G16H 10/60

(57) ABSTRACT

A system and method for monitoring and auditing the patient's health care compliances in order to detect bedside patient's information such as bed sores, oral health care, insertion of invasive devices, patient positions etc. The system employs visual sensors such as lidars, IR cameras etc. for observing the patients. The system also uses machine learning algorithms that use the bedside patient information into pre-trained models so as to monitor, trace and diagnose patient histories by the caretakers.

### CROSS REFERENCE TO RELATED APPLICATIONS

The present application derives priority from U.S. Pat. App. No. 63/242,063 filed on 9 Sep. 2021 and incorporated herein for reference.

### FIELD OF INVENTION

The present invention relates to a patient monitoring system, more particularly to the system and method for automating bedside infection audits using machine learning algorithm.

### BACKGROUND OF THE RELATED ART

With the development of technology, it is the need of the hour to automate complex task so that it aids the tedious task of different professional communities to make it simpler and easier. The heavy impact of artificial intelligence and related technologies are rapidly prevalent in business and society and the drastic technological impact has also emerged in the healthcare sector.

The emergence of artificial intelligence (AI) as a tool for better health care offers unprecedented opportunities to improve patient and clinical team outcomes, reduce costs, and impact population health. Examples include but are not limited to automation; providing patient, caregivers, and health care professionals' information synthesis; and recommendations and visualization of information for shared decision making. The application of machine learning had a huge impact in healthcare area and has reduced the complexities contributing effortless and precise patient care in the health care sector.

Compliance of daily bedside infection related protocols is a big visibility gap in US Hospitals resulting in over $80 billion+ in direct costs and $3 million+in-patient infections. The challenge is especially acute for patients with invasive devices such as catheters, ventilators and central lines. The state of the art using video intelligence top sensors such as Lidars/IR cameras is to detect bedside hygiene activities involving macro activities such as sleeping, walking, hand-hygiene & cleaning by caregivers. However, the accuracy of existing methods is limited by challenges such as occlusion while more complex and granular activities (such as various types of oral care on ventilated patients) are limited by inability of existing approaches to correctly classify such granular activities.

The present invention solves these challenges. It discloses a combination of visual sensors with physical placement optimized to reduce occlusion. The invention also discloses a system and method for catheter monitoring, bedside infection auditing and patient monitoring in the ventilator unit. In addition, it uses a novel approach to detect more minute level bedside information such as patient position, invasive devices on the patient with their positions, bedside objects etc. These parameters are further classified using a pre-trained model to also detect various macro activities such as hand-hygiene, cleaning, bed positioning, catheter cleaning, oral care, etc. The present invention uses a plurality of visual sensors such as Lidars/IR cameras in combination with machine learning algorithms to autonomously measure and classify various complex and minute bedside compliance activities related to infection prevention.

Many systems are available that supports in patient monitoring systems as application in the health or medical industry. Its application varies from healthcare monitoring systems which are related to healthcare sanitization, monitoring patient hygiene related compliance etc. Many of the available technology or system helps healthcare providers to monitor the bedside compliance activities using intelligent multi-function electronic caregiving system to facilitate advanced health diagnosis, health monitoring, fall and injury prediction, health maintenance and support, and emergency alerts.

There are various patents relating to the system and method for automated care giving assistance and healthcare monitoring and hand hygiene systems. Patent application, US 2018/0296413 A1, discloses the method of patient care device integration with a hospital bed. The invention specifically discloses integration of catheter monitor into patient support systems.

The granted patent U.S. Pat. No. 9,642,967 B2 reveals Catheter monitor integration system with patient support systems such as patient beds and with other healthcare communication systems including hand hygiene system

In another patent application, WO 2016/207370 A1, discloses hand hygiene compliance monitoring system. Patent application, US 2016/0267327 A1, discloses the method and system for monitoring a patient within a medical monitoring area with depth camera device. Further, the granted application, U.S. Ser. No. 10/813,572B2 discloses an intelligent system for multi-function electronic care giving to facilitate advanced health diagnosis, health monitoring, fall and injury prediction, health maintenance and support, and emergency response.

Most of the prior arts cited discusses about monitoring macro level activities such as hand hygiene, sanitization, patient sleep hours etc. The present invention instead not only automates the macro-level activities, it also monitors the patient's micro-level activities such as hand-hygiene, cleaning, bed positioning, catheter cleaning, oral care, etc. Further, the present invention also incorporates restricted machine learning pre-trained module that helps to predict and diagnose patient's health audits.

### SUMMARY OF THE INVENTION

The disclosed invention relates to the system and method of automating bedside infection audits using machine learning algorithm. Machine learning applications can readily cope with the dynamic nature of healthcare than traditional surveillance models which is why the present invention utilizes an innovative approach towards automating bedside infection audits. In one aspect of the invention, it describes the system of monitoring and auditing the patient's bedside infection and other minute level activities.

Another aspect of the invention discloses the system for monitoring patients that uses a combination of visual sensors, sound sensor, pressure sensor, temperature sensor, vicinity sensor, sanitizer dispensing sensor etc. with physical placement optimized to reduce occlusion. The other sensors utilize in the present invention may be a combination of but not limited to certain sensors.

In yet another aspect of the invention, it describes a novel method to detect granular bedside information such as patient position, invasive devices on the patient with their positions, bedside objects etc.

Yet another aspect of the present invention discloses the system for catheter and central line monitoring within the patient health auditing system. It also discloses a system of automating and observing patient care ventilation unit.

In yet another aspect of the present invention, the system employs machine learning technique by pre-trained models that uses data as the bed side parameters to detect various macro activities (such as hand-hygiene, cleaning, bed positioning, catheter cleaning, oral care etc) as well as micro activities (such as patient position, invasive devices on the patient with their positions, bedside objects etc.)

In another aspect of the present invention, it discloses a method that develops a pre-trained model for detecting macro-level and micro-level activities that are healthcare compliance related.

In yet another aspect of the present invention, the invention discloses a system that monitors and audits the patient's bedside infection parameters. The present invention uses LIDARs to enhance image texture and aids in improved spatial detection of objects around the Intensive Care Unit.

### BRIEF DESCRIPTION OF THE ACCOMPANYING DRAWINGS

FIG. 1 is a schematic view of an illustrative system for a networking service of the disclosure.

FIG. 2 illustrates an exemplary diagram of the system for monitoring and automating bedside infection information and activities.

FIG. 3 illustrates flowchart of the system and method for automating bedside infection audits using pre-trained modules.

FIG. 4 illustrates flowchart of the system and method for monitoring micro activities.

FIG. 5 illustrates a diagram of the patient monitoring system.

FIG. 6 illustrates a general architecture of remote patient's monitoring system.

FIG. 7 illustrates a diagram of analyzing hand hygiene.

FIG. 8 illustrates a flowchart of activating hygiene protocol.

FIG. 9 illustrates a block diagram displaying arrangement of LIDARs, sensors and IR cameras.

Other aspects of the present invention shall be more readily understood when considered in conjunction with the accompanying drawings, and the following detailed description, neither of which should be considered limiting. Each of the objects stated above will be described in further detail in the next sections.

### DESCRIPTION OF THE PREFERRED EMBODIMENTS

Unless otherwise defined, all terms (including technical and scientific terms) used herein have the same meaning as commonly understood by one of ordinary skill in the art of this disclosure. It will be further understood that terms, such as those defined in commonly used dictionaries, should be interpreted as having a meaning that is consistent with their meaning in the context of the specification and should not be interpreted in an idealized or overly formal sense unless expressly so defined herein. Well known functions or constructions may not be described in detail for brevity or clarity.

The terminology used herein is for the purpose of describing particular embodiments only and is not intended to be limiting. As used herein, the singular forms “a”, “an” and “the” are intended to include the plural forms as well, unless the context clearly indicates otherwise.

With reference to the use of the words “comprise” or “comprises” or “comprising” in the foregoing description and/or in the following claims, unless the context requires otherwise, those words are used on the basis and clear understanding that they are to be interpreted inclusively, rather than exclusively, and that each of those words is to be so interpreted in construing the foregoing description and the following claims.

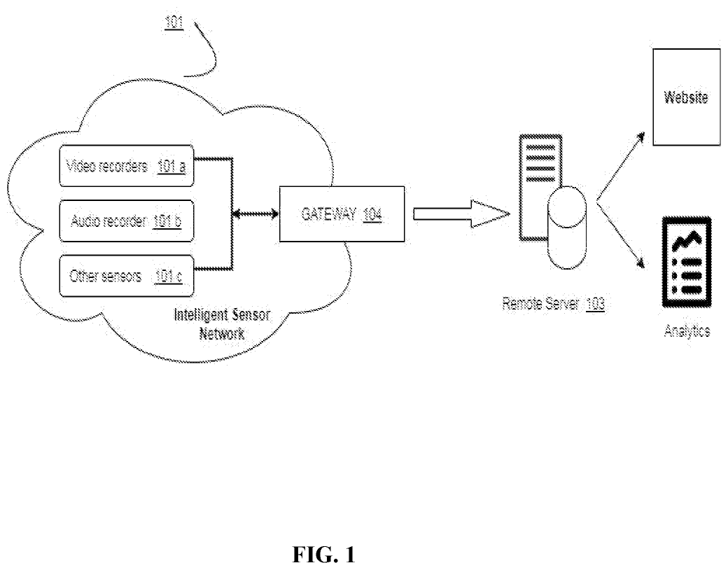

The invention discloses the system and method for automating bedside infection audits using machine learning. FIG. 1 is a simplified system diagram of networking service of the invention disclosure. The system 100 has an intelligent sensor network 101, which is a surveillance unit coupled with video sensors (101a), audio recorders (101 a), and other sensors (101 c) to monitor the patient's health. The system data is processed in a remote server (103) wherein the patient health data and information can be analyzed and monitored by the caregivers or healthcare professionals from any remote system. The monitored data is used for automating the bedside infection audits and helps the health care professionals to act timely.

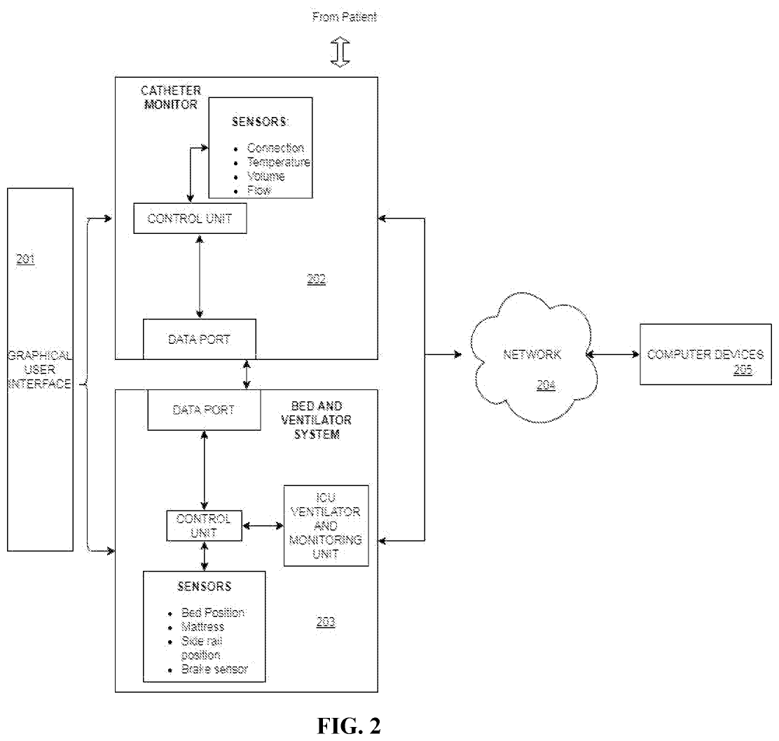

FIG. 2 illustrates an exemplary diagram of the system for monitoring and automating bedside infection information and activities. The system depicts a graphical user interface module (201) which is accessible to the healthcare professional. It extracts data from a catheter monitoring module (202) and a bed control and ventilator module (203). Each of these modules comprises a control unit which controls various sensors and data/power ports. For e.g. it monitors the temperature, volume and flow rate of the urine through the Catheter monitor and devices. The data from the catheter, bed control and ventilator monitoring systems further may be accessed by various computing devices (205) over the connecting network (204).

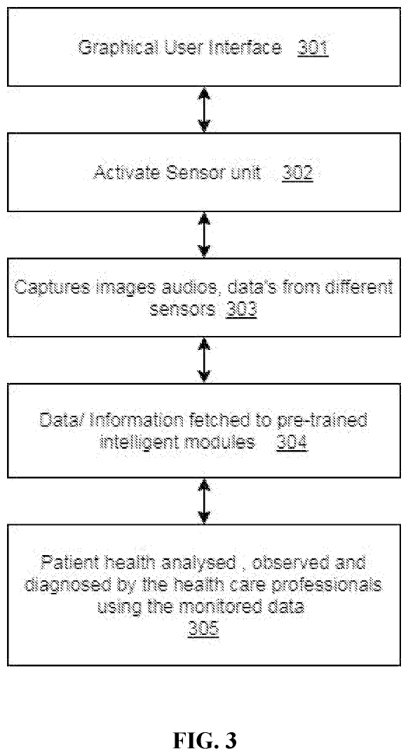

FIG. 3 illustrates a basic flowchart of the invention disclosed. The system has a graphical user interface (301) for which the system enables various sensor units (302) for monitoring the catheter and the passage of the fluid from/into the body. It captures images, audios and micro-level patient data's from different sensors (303). The data or information monitored is fetched to pre-trained intelligent modules (304). These patient health data is further used, analyzed, and diagnosed by the health care professionals using the monitored data (305). The overall system automates the bedside infection audits using machine learning algorithm. The machine learning algorithm enables monitoring remote patients and alerts their current health condition. The present invention offers contactless observation demonstrating that it is only necessary for the patient to be present within a few meters of the sensors for them to work. Further, various sensors in the present invention identify the symptoms of the patients, followed by machine learning algorithms processing the symptoms data sets and identifying the disease. The information is further transmitted to the health care professionals for further assessment.

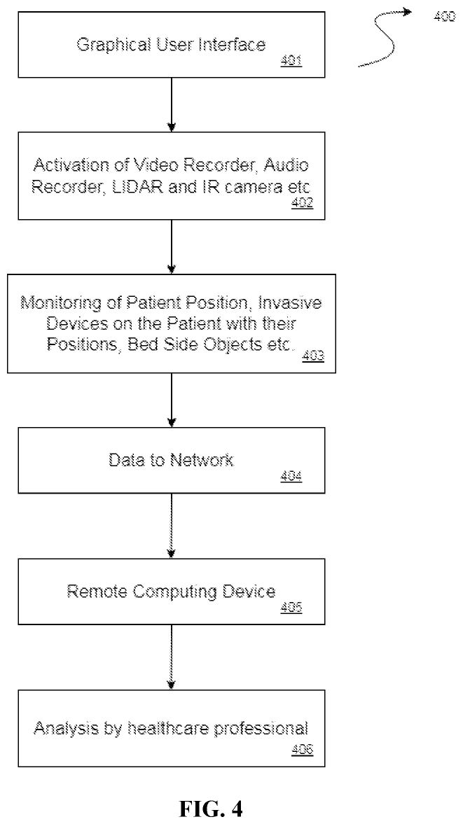

FIG. 4 illustrates a flowchart of the system and method for monitoring micro activities. With the help of graphical user interface (401) the healthcare professional activates various sensors like video recorder, audio recorder, LIDAR and IR camera, and position sensors etc (402). These sensors monitors patient position, invasive devices on the patient with their positions, bed side objects, etc (403). For example the IR camera and LIDAR, video recorder provides visual data of patient and bedside parameters. Similarly, position sensor monitors backrest elevation within hospital bed. All these information are stored in the form of images, recorded audios and videos (404). This information can be retrieved or analyzed by any remote computing device (405) which is connected to the system network by healthcare professional. On the basis of data the healthcare professional takes further decisions (406).

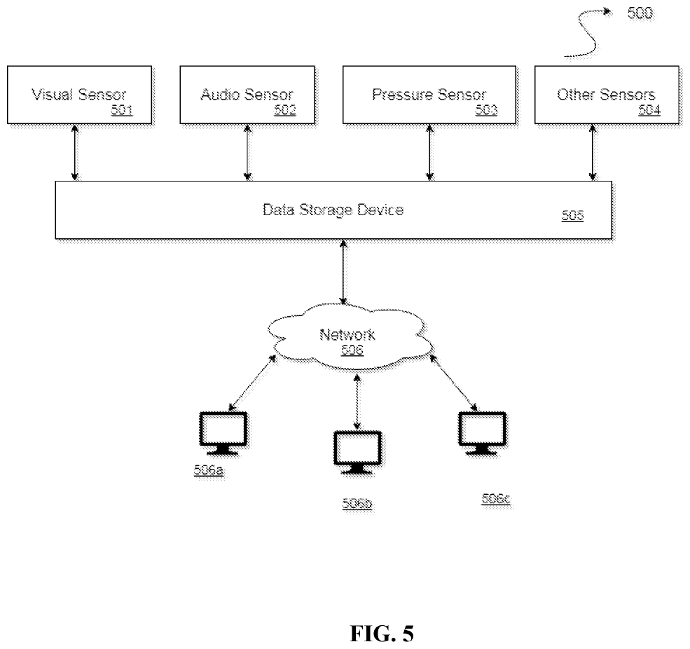

FIG. 5 illustrates a simple diagram of the patient monitoring system. According to present invention, the system as illustrated in FIG. 5 comprises different sensors including visual sensor (501), audio sensor (502), pressure sensor (503), temperature sensor, position sensor, vicinity sensor etc. All these sensors monitor physiological information of the patient. For example visual sensor (501) including camera and video recorder captures images of patient and all the bedside objects, video recorder records the video of patient and patient's surrounding, temperature sensor monitor the temperature of patient, pressure sensor (503) monitors patient breathing, detects when the patient inhales and exhales and position sensor monitors backrest elevation within hospital bed. All the parameters like pressure, temperature, position and patient's bedside information stored into the data storage device (505). The healthcare professional can obtain all the physiological information of patient from analyzing these parameters through various devices (506a, 506b and 506c) remotely which is connected to the network.

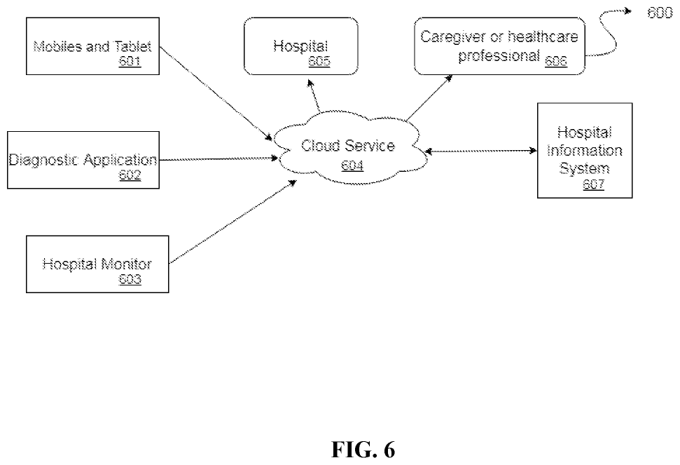

FIG. 6 illustrates a general architecture of remote patient's monitoring system. The remote patient's monitoring system (600) according to the present invention comprises computing device like mobiles and tablet (601), diagnostic application (602), hospital monitor (603), hospital (605), caregiver or healthcare professional (606) and hospital information system (607). All these components of remote patient's monitoring system (600) are connected to the cloud service (604). All the information of patient's physiological parameters, patient's hygiene data, hospital's hygiene, patient's records like all the diagnostic tests that are carried out and given treatment are stored into the cloud service (604). All these information is monitored and analyzed by hospital information system (607) and caregiver or healthcare professional (606). The remote patient's monitoring system (600) further includes a combination of various sensors like visual sensors, sound sensor, pressure sensor, temperature sensor, vicinity sensor, sanitizer dispensing sensor etc to monitor patient, patient's surroundings and hospital. The caregiver or healthcare professional (606) analyzes data remotely through cloud service (604) and runs diagnosis, treatment or hygiene protocol accordingly.

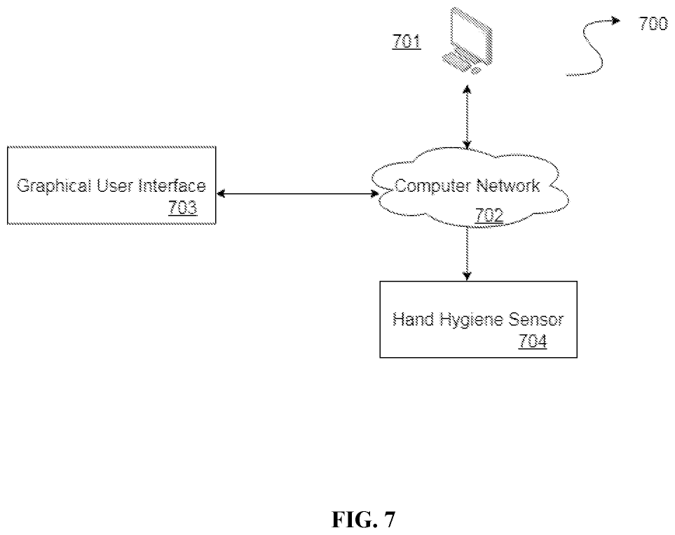

FIG. 7 illustrates a diagram of analyzing hand hygiene. As illustrated in the system (700) may comprise a computing device (701), computer network (702), graphical user interface (703) and hand hygiene sensor (704). The computing device (701), graphical user interface (703) and hand hygiene sensor (704) are connected to the computer network (702). The hand hygiene monitoring system utilizes a machine learning algorithm to achieve hand hygiene goal. The hand hygiene monitoring system (700) monitors patient and patient's bedside objects and transmit this data to computer (701) through a network (702). The data like hand hygiene records, images of patients and recorded videos are analyzed by caregiver or healthcare professionals.

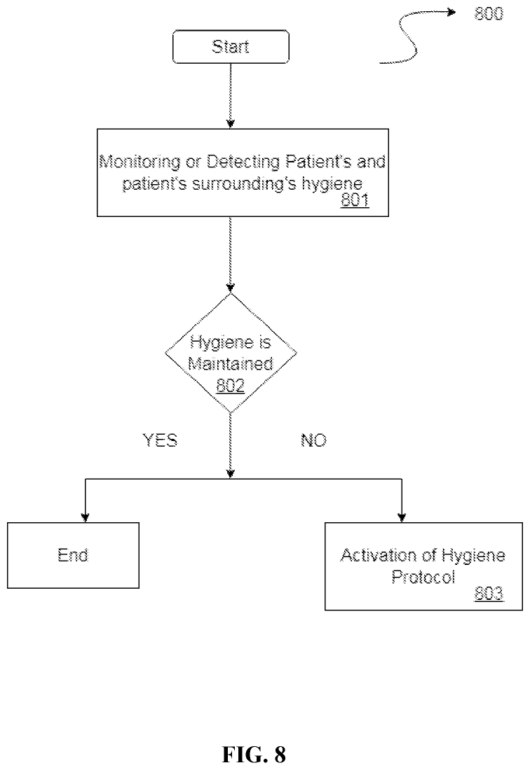

FIG. 8 illustrates a flowchart of activating hygiene protocol. One of the embodiments of the present invention runs hygiene protocol. The system (800) which monitors hygiene of the patient and patient's surrounding comprise different sensors including visual sensor, sound sensor, pressure sensor, temperature sensor, vicinity sensor, sanitizer dispensing sensor etc. The other sensors utilize in analyzing hygiene level may be a combination of but not limited to different sensors. By utilizing a combination of different sensors the system (800) monitors personal hygiene of patient along with the patient's surrounding's hygiene. After analyzing all the hygiene parameters the machine learning algorithm decides whether the hygiene is maintained or not. If hygiene parameters do not meet the required criteria the system (800) activates hygiene protocol. The hygiene protocol includes floor cleaning, table cleaning, spraying and object cleaning etc.

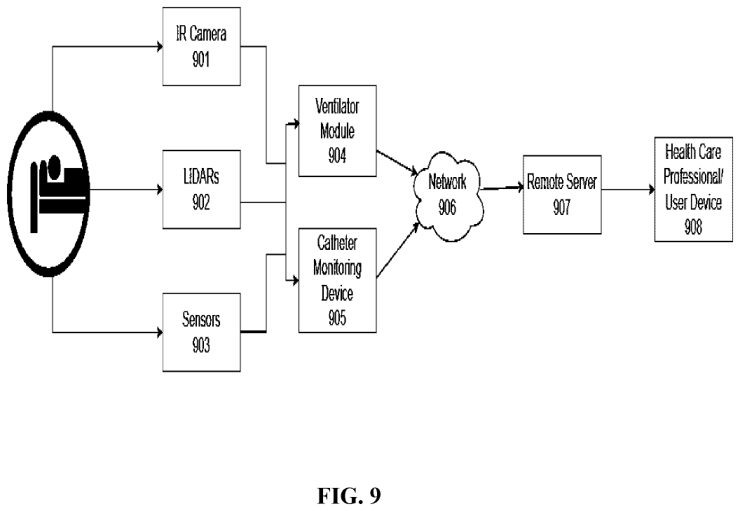

FIG. 9 illustrates a block diagram of the present invention displaying a system (900) comprising IR (Infra Red) cameras (901), LIDARs (902) and various other sensors (903) including but not limited to visual sensors, sound sensors, pressure sensors, temperature sensors, vicinity sensors, position sensors and sanitizer dispensing sensors which are monitoring the patient and the data thus collected is sent to the ventilator module (904) and catheter monitoring module (905) which further extracts patient's bed side information or data provided by the IR cameras (901), LIDARs (902) and various sensors (903). The data from the Ventilator module and catheter monitoring module is further sent to a remote server (907) over the network (906) from where it may be accessed by the health care professional/user device (908). The present invention uses LIDARs (laser imaging, detection, and ranging) to enhance scanned image (3D) texture and aids in improved spatial detection of objects around the Intensive Care Unit more precisely Patient's bedside movements and Bedsore Management.

What is claimed is:

1. A method comprising:
a. automating bedside infection audits using machine learning algorithm;
b. monitoring and auditing patient's granular bedside parameters including macro and micro activities;
c. machine learned pre trained models that use data as the bed side parameter to detect various macro and micro activities;
d. automating and observing patient care ventilation unit.

2. A method according to claim 1, wherein the macro activities may include but not limited to hand-hygiene, cleaning, bed positioning, catheter cleaning, oral care of the patients.

3. A method according to claim 1, wherein the micro activities may include but not limited to patient's position, invasive devices on the patient with their positions, bedside objects.

4. A method according to claim 1, wherein the bedside infection audit utilizes machine learning algorithm.

5. A system comprising:
a. an intelligent sensor network, LIDARs and power ports;
b. a surveillance unit coupled with video sensors, audio recorders, and other sensors to monitor the patient's health;
c. a remote server wherein the patient's health data and information can be analyzed and monitored by the caregivers or healthcare professionals;

6. A system according to claim 5 further comprising:
a. a graphical user interface module;
b. a bed control and ventilator module;
c. a catheter monitoring module;
d. a control unit which controls various sensors, LIDARs and power ports.

7. A system according to claim 6, wherein the catheter monitoring module monitors the temperature, volume and flow rate of the urine through the catheter monitoring device.

8. A system according to claim 6, wherein the data from the catheter, bed control and ventilator modules further may be accessed by various computing devices over the said connecting network.

9. A system according to claim 8, wherein the data from the catheter, bed control and ventilator module is analyzed and diagnosed by the health care professionals.

10. A system according to claim 5, wherein the sensor network may comprise of but not limited to visual sensors, sound sensors, pressure sensors, temperature sensors, vicinity sensors, position sensors and sanitizer dispensing sensors.

11. A system according to claim 5, wherein the remote server has access to data sets including but not limited to patient's hygiene data, hospital's hygiene data, patients records, diagnostic tests.

12. A system according to claim 11, wherein the information of data sets is monitored and analyzed by the hospital information system routinely.

13. A system according to claim 6, wherein LIDARs aid in monitoring the bed control, catheter and ventilator modules by scanning objects around the Intensive Care Unit more precisely Patient's bedside movements.

14. A system according to claim 6, wherein LIDARs are used to enhance the scanned image texture and aids in improved spatial detection of objects around the Intensive Care Unit more precisely Patient's bedside movements and bedsore management.
